(19) United States
(12) Patent Application Publication
(10) Pub. No.: US 2013/0220297 A1
(43) Pub. Date: Aug. 29, 2013

(54) Heat Generating Single-Use Garment

(71) Applicants: Milos Sivucka, Rancho Cordova, CA (US); Miroslava Sivucka, Rancho Cordova, CA (US)

(72) Inventors: Milos Sivucka, Rancho Cordova, CA (US); Miroslava Sivucka, Rancho Cordova, CA (US)

(21) Appl. No.: 13/763,973

(22) Filed: Feb. 11, 2013

Related U.S. Application Data
(60) Provisional application No. 61/603,071, filed on Feb. 24, 2012.

(51) Int. Cl. A61F 7/03

(52) U.S. Cl. CPC A61F 7/034
USPC 126/204

(57) ABSTRACT

Disclosed is an emergency cold weather heating garment that is adapted for one-time use when rapid heating is required to prevent hypothermia and exposure. The garment comprises an article of clothing that comprises a plurality of enclosed pouches having a chemical heating agent therein. The garment is adapted to be deployed from a vacuum sealed container or wrap, and once deployed the chemical heating pouches are air-activated, initiating an exothermic reaction that gives off heat and raises the body temperature of the wearer. Each pouch comprises an air-permeable or perforated exterior surface, an interior volume of solid, air-activated heating material, and an underside surface that conducts heat across its surface. The garment is adapted to be deployed and donned above or below other clothing articles for rapidly heating the user over a period of time as the chemical heating material reacts.

### CROSS REFERENCE TO RELATED APPLICATION

This application claims the benefit of U.S. Provisional Application No. 61/603,071 filed on Feb. 24, 2012, entitled “Survive an Accident in Wilderness.” The above identified patent application is herein incorporated by reference in its entirety to provide continuity of disclosure.

### BACKGROUND OF THE INVENTION

1. Field of the Invention

The present invention relates to heated articles of clothing and emergency heating covers. More specifically, the present invention pertains to an emergency, single-use garment that includes air-activated chemical heating means that rapidly warms a user in cold climates and in emergency situations.

Many individuals enjoy outdoor winter sports or activities where they may be exposed to extremely cold environments for prolonged periods. Those who enjoy hiking and camping excursions expose themselves to outdoor elements. In higher elevations or colder climates, these outdoor elements may include environments with rapid temperature fluctuations that drop well below freezing during certain periods of the day. Adequate clothing that keeps individuals both warm and dry is imperative to remaining healthy and preventing bodily injury in these environments. Under normal conditions, the human body maintains its core temperature through the process of thermoregulation, where heat is generated within the body and warm blood is pumped throughout the body's extremities to maintain a narrow body temperature range. In cold and damp environments, an individual's body temperature can drop below healthy temperature ranges and into a hypothermic state. Prolonged hypothermia can reduce mental capacity and bodily functions, result in physical exposure damage, and further lead to serious, life threatening conditions if not treated. Those engaging in winter sports and outdoor activities, including those in remote locations and those without a ready means of staying warm or generating heat, are prone to be exposed to hypothermic conditions that associated hazards therewith.

To combat the cold climate, users generally wear layers of clothing and extremity coverings to prevent core temperature fluctuations and frost bite. Over these layers may further be donned a moisture shell that prevents water from infiltrating the garment layers and reducing their effectiveness. In very cold conditions, an external heat source may be required to assist the body maintaining its core temperature. Common heat sources include camp fires, hand warmer devices and personal heating appliances. However, these heating means are not appropriate or feasible in all conditions. If one is stranded or deployed into a harsh environment, an alternative source of heat may be required to keep warm. In emergency situations, rapid application of heat may also be required to elevate an endangered individual's body temperature to prevent long-term damage.

One commonly deployed heat source in camping and outdoor excursions includes chemical heating packs. These heating packs are enclosed heat-generating means that utilize an exothermic chemical reaction based in the presence of a catalyst to generate heat for the user. The generated heat is transferred to the individual's body to warm the user for a given period as the chemical reaction occurs. Such devices are common in hand and foot warmer packs for the purposes of preventing frost bite. Most of these disposable warmers generate heat by a process that creates iron oxide, or rust. The pouches typically contain an iron powder, a salt, water, an absorbent material, and activated carbon. When the hand warmer pouch is exposed to the environment, oxygen crosses the permeable covering of the warmer pouch to react with the compound therein. With salt and water present, the oxygen reacts with the iron powder to form an iron oxide in an exothermic reaction that releases heat from the pouch, thereby warming a portion of the user's body in contact with the pouch without resorting to an open flame or an electrical heating appliance.

The present invention pertains to a single-use garment that comprises a plurality of air-activated chemical heating pouches disposed about its surface to create a readily donned covering that surrounds and rapidly warms a user. The garment is deployed from a sealed container or wrapping, whereafter oxygen crosses a permeable boundary forming an exterior surface over each pouch to initiate a chemical reaction that exposes heat. The device comprises a garment that readily wraps around a user, where the device may be deployed in a number of garment styles. The device is ideal of winter sports enthusiasts, those in emergency situations where no external heat source is present, military use, or for those responding to an emergency where rapid heat application to a patient is required. The garment is adapted to rapid deployment and one-time use, whereafter it can be kept donned or thereafter disposed of in favor of a replacement. The garment can be worn below or over existing clothing layers such that the garment acts as an additional heating garment while the additional layers of clothing can maintain the user's body heat after the reaction has ceased.

2. Description of the Prior Art

Devices have been disclosed in the prior art that relate to heated garments and those with internal compartments for housing heating elements. These include devices that have been patented and published in patent application publications, and generally relate to multiple use garments for supporting heating means. The following is a list of devices deemed most relevant to the present disclosure, which are herein described for the purposes of highlighting and differentiating the unique aspects of the present invention, and further highlighting the drawbacks existing in the prior art.

Specifically, U.S. Pat. Nos. 5,302,806 and 5,605,144 to Simmons disclose a reversible vest having a front and rear panel adapted to attach to a user's torso in and apply a heating packet to either side of a user's torso. The vest panels are positionable against the user's body facing either direction to apply heat to the front or rear of the user's torso. The vest includes an inner and outer adjacent layer that form a pouch for securing heating packets thereinto, while the inner layer that faces the user's body is a porous layer to allow heat transfer. Alternate embodiments of the Simmons device contemplate a cloth head band and a general garment that support an inserted air-activated chemical heating packet. Unlike the Simmons devices, the present invention is focused on a single-use, emergency situation heating garment that does not contemplate replaceable heating packets, but rather has air-activated chemical pack pouches deployed about the garment's structure. The garment is removed from its sealed container and begins reacting with the air through the porous or vented heating element supports, whereafter the garment is donned for rapid heating of the user's body. The garment is disposable thereafter or further garments may be deployed as necessary.

U.S. Published Patent Application Publication No. 2008/0029080 to Dodo discloses an active iron powder composition that generates heat upon contact with the air to warm a user's body. Different compositions are disclosed and containers for securing the air-activated substance. The composition is housed within a heat generating body having a thickness that can be applied to the human body for warmth. The Dodo device, while disclosing a heating composition and housing therefore, fails to disclose the novel features of the present invention. Notably, the present invention contemplates the use of a chemical heating composition that is air-activated, but is primarily concerned with the structure and implementation of the chemical heating means in emergency and single-use situations where a user requires rapid body heating.

Finally, U.S. Pat. No. 6,185,744 to Poholski discloses a personal thermal garment comprising a shell and a thermal insert that is adapted to cover the back and chest portion of a wearer. The thermal insert is one that is preheated by the user or cooled prior to being placed into the garment for heating or cooling the user. The shell includes an inner and outer layer that defines a chamber for accepting the thermal insert therein. The outer layer prevents heat from transferring across its section while the underside layer facilitates heat transfer to allow the heating or cooling effects to directly apply to the user's body rather than escaping into the environment. While the Poholski device describes a compartmentalized garment for holding a heating element, the Poholski device does not contemplate a single-use garment and a heat generating material that reacts with the ambient air once deployed. The present invention does not require preheating or cooling of an internal element, by contrast the present invention is designed for those situations in which heat is desperately required and no such heating source is otherwise present. The Poholski device is more aptly designed for use in less desperate situations.

The present invention provides a new and novel heating garment that is adapted for one-time and emergency use in low temperature environments. The device is distributed within a vacuum-sealed container or bag such that the garment may be easily stowed for emergency use, whereafter it can be withdrawn from the pack to initiate the heating process and rapidly donned. It is submitted that the present invention is substantially divergent in design elements from the prior art, and consequently it is clear that there is a need in the art for an improvement to existing garments with active heating means. In this regard the instant invention substantially fulfills these needs.

### SUMMARY OF THE INVENTION

In view of the foregoing disadvantages inherent in the known types of heated garments now present in the prior art, the present invention provides a new, single-use garment having heating means disposed about its surface such that the device can be utilized for providing convenience for the user when in very cold or harsh environments or deployed in emergency situations.

It is therefore an object of the present invention to provide a new and improved heated garment device that has all of the advantages of the prior art and none of the disadvantages.

It is another object of the present invention to provide a heated garment that comprises a plurality of pouches disposed about its outer surface, whereby each pouch contains an oxygen-activated composition that generates heat when the garment is exposed to the air.

Another object of the present invention is to provide a heated garment device that is packaged within an air tight and vacuum sealed container prior to use, whereafter the device is exposed to the atmosphere when deployed to initiate the heat generation reaction.

Yet another object of the present invention is to provide a heated garment device that is adapted for one-time use and is disposable thereafter.

A final object of the present invention is to provide a garment device having several styles such that the garment provides coverage over different parts of a user's body when donned, including a garment top, a bottom, a shoe covering, a hand covering, and a head covering.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a view of the present invention in a stowed state within its vacuum-sealed packaging prior to use.

FIG. 2 shows the heated garment being deployed and the heat pouches reacting with the atmosphere.

FIG. 3 shows a top cover embodiment of the present invention.

FIG. 4 shows another top cover embodiment of the present invention.

FIG. 5 shows a cross section of the heated pouches.

FIG. 6 shows the different style garments contemplated by the present invention.

### DETAILED DESCRIPTION OF THE INVENTION

Reference is made herein to the attached drawings. Like reference numerals are used throughout the drawings to depict like or similar elements of the heated garment device. For the purposes of presenting a brief and clear description of the present invention, the preferred embodiment will be discussed as used for rapidly heating a user in cold climates by providing a single-use, deployable garment chemical heating means disposed thereon. The figures are intended for representative purposes only and should not be considered to be limiting in any respect.

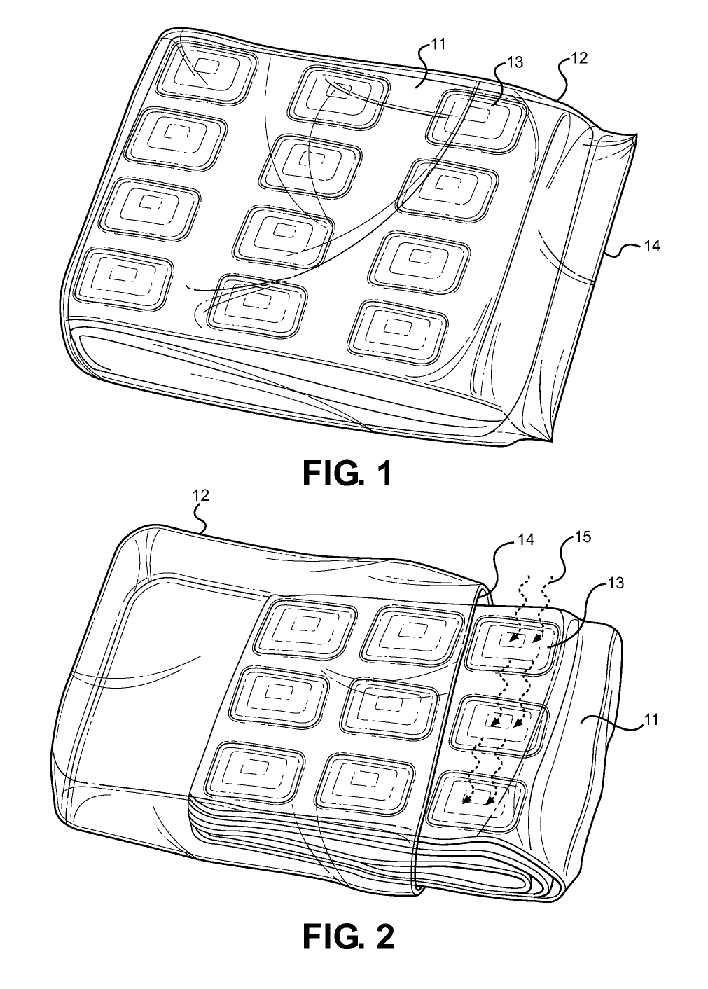

Referring now to FIG. 1, there is shown a view of the present invention in a stowed state prior to use. The present invention comprises a single-use garment that is deployable from a vacuum-sealed wrapping 12 or vacuum-sealed container. The wrapping 12 has its interior volume evacuated of air to store the garment therein without activating the heating means, whereafter the wrapping is opened to expose the garment to the environment. The wrapping remains sealed until the garment is used and further contains no penetrations to the outside air. The garment 11 itself comprises an article of clothing having a plurality of air-activated heating means 13 disposed about its surface, such that when the garment 11 is withdrawn from its packaging 12 and exposed to the atmosphere, the heating means reacts with oxygen in the air to generate heat. The packaging includes a tear-open seal 14 or similar means of accessing the garment 11 therein.

The garment is a disposable article of clothing that is specifically designed to heat a user in cold climates, either in an emergency situation or where no other suitable heating means is available. The heating means comprises a plurality of pouches 13 disposed about the garment surface, within which is contained a chemical heating agent that is adapted to react with oxygen in the air in an exothermic reaction that generates heat. This heat is transferred to the user's body for warmth and to fight exposure and hypothermia. The exact style of garment may take several forms, including variations in type of garment, its design elements, and the array of heating elements disposed thereon. The different garment types provide warmth to different parts of the body while the array of heating elements varies the magnitude of heat given off by the garment and thus desired by the user (i.e. greater or fewer heating pouches raises or lowers the heating capacity of the garment).

Referring now to FIG. 2, there is shown a view of present garment 11 being deployed from its stowed state within the vacuum-sealed wrapping 12 through the unsealed opening 14 thereof prior to use. The heat generating pouches 13 of the garment 11 are adapted to allow air to permeate 15 across their outer surface such that the chemical compound therein can initiate its reaction to produce heat. The placement of the heat pouches about the surface of the garment allows heat to be generated in multiple locations around the user's body, while the chemical reaction is one that initiates rapidly and lasts for a significant period of time to provide rapid and prolonged heat for the user. In its contemplated form, the present invention is packaged and shipped within the vacuum-sealed wrapping 12 such that the assembly can be stowed within a backpack or other suitable carrier, and thereafter deployed when the user requires an additional heating source. The garment itself is adapted to rapidly deploy from its packaging, where the seal opening 14 may comprise a tear-away seal or similar rapidly-opening seal, granting access to the garment to then be donned by the user. It is contemplated that the garment is used only once and thereafter disposed of after the heating means have ceased their reaction and ceased producing heat. The garment can then be replaced or discarded as desired.

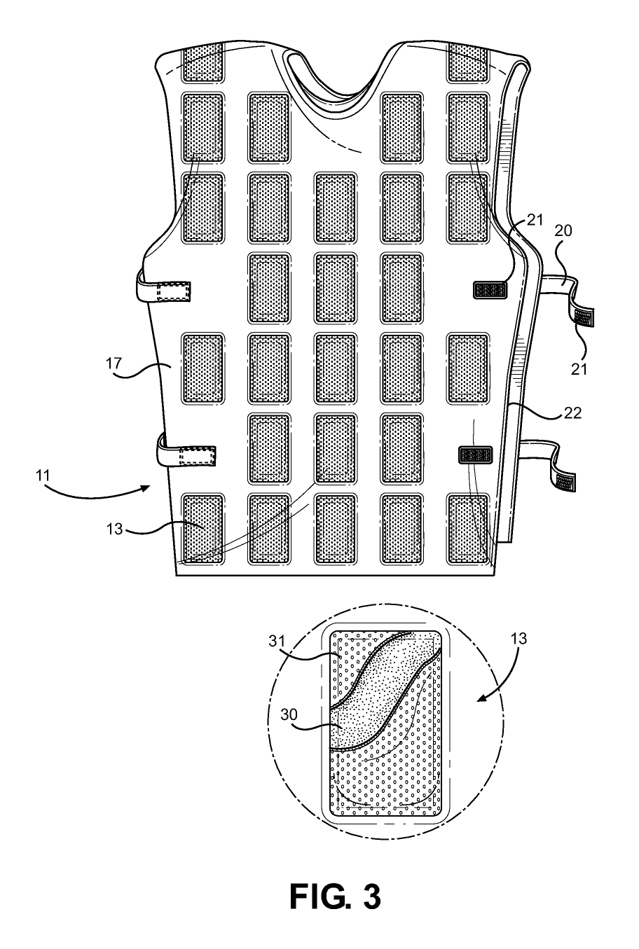

Referring now to FIG. 3, there is shown an upper body vest embodiment of the present garment and a close-up view of an individual heating pouch 13 thereon. In this embodiment, the garment comprises a vest 11 having a front and rear body portion 17 connected over the shoulders of a wearer that is adapted to cover a wearer's upper body region. The device is formed of a front side and a back side connected by a first and second shoulder portion. Along the exterior surface of the vest is a plurality of air-activated heating means contained within the pouches 13. The pouches are provided in a pattern across the garment to increase the heated surface area of the wearer. In a preferred embodiment of the vest, the garment front and rear body portion are disconnected below the wearer's arms, whereby strap connectors 20 allow the garment to be secured rapidly about the user's torso and over existing clothing if desired. The straps 20 extend from the backside toward the front side of the garment and are removably connected along the lateral sides 22 of the garment using a corresponding connection means 21. This may include hook and loop fasteners, snaps or other suitable connector elements that allow the strap to secure the lateral portions of the garment about a user for looser or tighter fitment. The open configuration allows a wearer to easily don the garment over existing clothing, accommodating the thickness of the clothing and different sized users.

Each of the heat pouches 13 comprises an exterior surface 31, an interior volume containing a chemical composition 30, and a backside surface facing inward from the garment. The exterior surface 31 comprises a perforated or air-permeable layer that allows air to pass therethrough and into the interior volume. When the garment is exposed to the environment, the chemical compound reacts with the air to generate heat, which in turn warms the user for a period of time.

The chemical within each pouch is preferably a solid material that is reactive with oxygen to produce an exothermic reaction. The present invention contemplates several embodiments for this chemical, which is well understood in the art of hand warmer devices. The present invention offers a disposable garment deploying a plurality of hand warmer-style pouches disposed about its surface for emergency and one-time use, where the device is provided within a sealed wrapping prior to use such that the user can travel with the garment and deploy it when required. The most common hand warmer compound used for generating heat is iron powder in combination with salt, water, an absorbent material and activated carbon. Oxygen in the air crosses the outer surface of each pouch to react with the iron powder in the presence of the salt and water to create iron oxide. This reaction produces heat and thus warms the user through a chemical reaction once the garment is donned. It is not desired to limit the present invention to a particular compound or type of pouch, but rather to disclose a new and novel means of deploying such heat means via a heated, single-use garment.

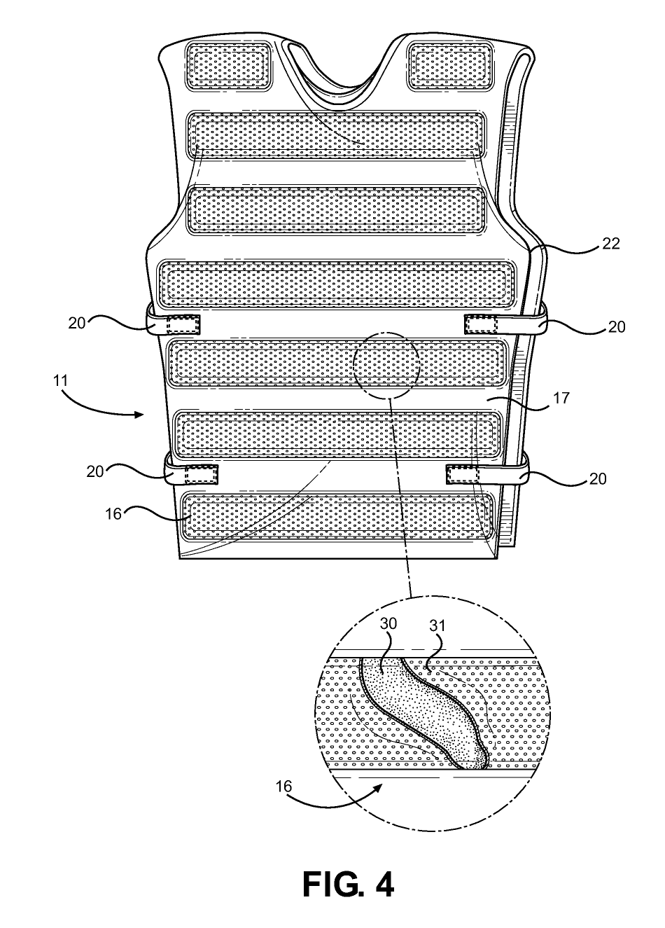

Referring now to FIG. 4, there is shown an alternate embodiment of the present invention, where the pouches are extended across the body section 17 of the garment to increase the heated surface area about the user. In this embodiment, the pouches are elongated strips 16 and extend laterally across the garment, as opposed to being smaller, more discrete pouches. The elongated strips include an open interior volume 30 and an outer, air permeable layer 31 in the same fashion as the pouches. Furthermore, the strips 16 may include stitching that segments the strip interior into shorter segments in order to keep the powder composition equally distributed across its length, or alternatively the strips 16 may include a single, open volume. The garment, as with the preferred embodiment, may take several different designs and preferably is a largely open structure that is secured about a user's body using strap connectors 20 that secure the edges 22 of the garment together.

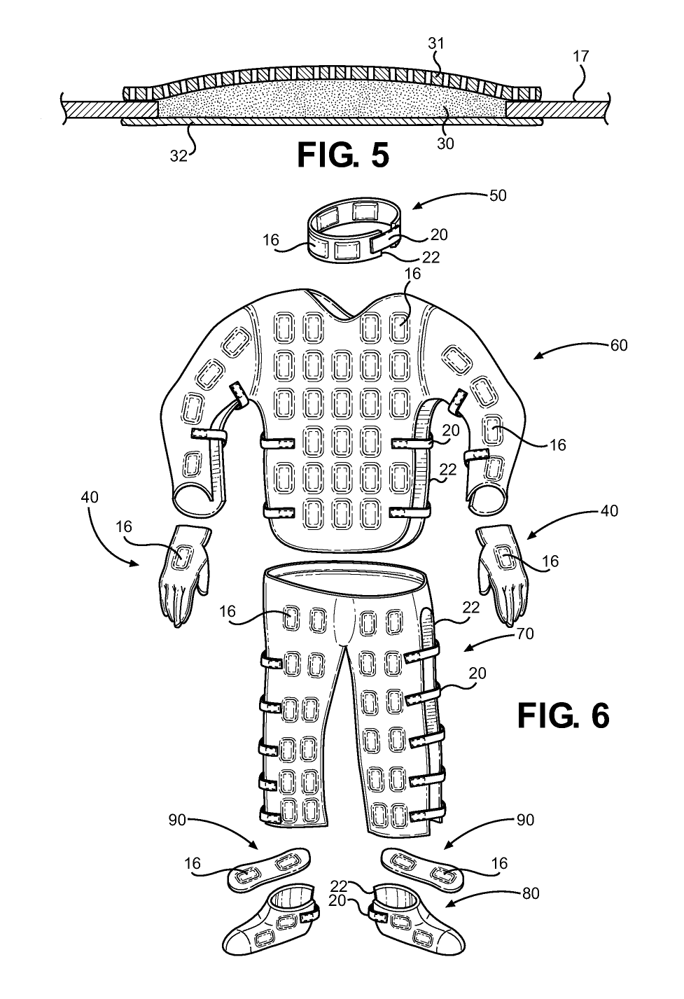

Referring now to FIG. 5, there is shown a cross section view of the heating means of the present invention. The heating means comprises a pouch or a strip that supports and contains the garment heating composition. The composition is sandwiched between two opposing layers such that oxygen from the air can enter the pouch interior and initiate the reaction, wherefrom heat from the reaction is transferred to the user. Specifically, the pouch comprises an outer, air-permeable layer 31, an interior volume filled with the powder heating composition 30, and an underside layer 32 that is adapted to transfer heat therethrough and towards the user. Integration of the pouch with the garment surface 17 may take several forms. As shown in FIG. 5, the inner 32 and outer 31 layer sandwich the garment 17, whereby the layers are stitched together about the periphery of the pouch. Alternate configurations are contemplated, including a stacked formation whereby the inner 32 and outer layer 31 of the pouch is attached under the garment layer or vise versa. The exact design of this interface is not desired to be limiting, but rather it is desired to disclose a pouch having an interior volume of powder reactant, a conductive inner layer that readily transfers heat to the user, and an outer layer 31 that allows air to permeate its thickness while containing the powder within the pouch.

Referring now to FIG. 6, there is shown several embodiments of the present invention that are designed to cover different portions of the wearer's body. The present invention contemplates a single-use garment having a garment layer or substrate upon which is supported a plurality of heating pouches or strips, whereby the garment is supported within a sealed wrapping prior to use and thereafter releases heat when donned. As shown, a garment upper body covering 60, lower body covering 70, shoe coverings 80, shoe sole inserts 90, hand coverings 40, and a headband or headgear element 50 is contemplated. These garments are adapted to be rapidly donned over a wearer's clothing, over body parts, or within the shoes of a user. Once removed from the packaging, the pouches 16 rapidly begin to produce heat, which is absorbed by the body to warm the user in that region. The body garments include open sides 22 or slits that secure together via strap elements 20 around the user to form a complete garment shell. The upper body garment 60 may be a pullover garment or a vest, which further may include open sides and arm lengths to allow for rapid donning directly over clothing without requiring the user to slide his or her arms through armholes. The garment surface can be applied directly over the wearer's existing clothing and secured thereto using the strap securement means 20. The headband 50, lower body covering 70, and the shoe coverings 80 also secure in a similar pullover or strap connection fashion. The strap connection means secures over a pair of disconnected edges formed in the particular garment that separates a front and rear portion of the upper and lower body embodiments, the rear of the shoe coverings, and the rear of the elongated headband embodiment. The shoe sole inserts 90 have an elongated body structure that is inserted below a user's feet and within a shoe article to heat the user's feet. Finally, the user's hands are warmed after the body portion and finger portion of the hand coverings 40 are donned over the user's hands and fingers, or alternatively over existing gloves.

Many people enjoy outdoor winter sports, hiking, camping, and other outdoor activities. These people typically protect themselves from the cold with layers of clothing and warm jackets. This outerwear can provide warmth, but if the temperature drops too low and the individual does not have access to a heating source, the individual may be at risk for exposure and hypothermia. Accidents while in the wild, prolonged winter events, being stranded, or further being deployed in a military operation can expose a person to extreme temperature with little options for remaining warm. Still others may be very sensitive to cold weather and require active heating sources to stay warm. For these conditions, the present invention provides a readily deployed and actively heated garment that utilized a plurality of chemical heating elements to produce heat and elevate the wearer's body temperature. The chemical heating elements are supported on a garment material substrate that comprises a thin, disposable material such as cotton or plastic. This allows the garment to be condensed into the sealed wrapping and be ready disposable after its use.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1) A heat producing, single-use garment, comprising:
an article of clothing having a body portion forming a surface;
said clothing surface having at least one heat generating means thereon;
said heat generating means comprising an enclosed portion of said surface comprising an exterior layer, an underside layer and an interior volume;
said enclosed portion interior volume having an air-activated heating compound that generates heat when exposed to air;
said enclosed portion exterior layer comprising an air-permeable layer;
said enclosed portion underside layer comprising a heat conductive material;
said article of clothing adapted to be deployed from a vacuum-sealed carrier, whereby said air-activated compound releases heat through said heat conductive layer when exposed to air for a period of time, whereafter said article of clothing being disposable after a single use.

2) The device of claim 1, wherein article of clothing comprises an upper body vest having a front side and a back side connected by a first and second shoulder portion.

3) The device of claim 2, wherein:
said vest front and back side further comprises disconnected edges;
said disconnected edges having a strap and strap connection means for securing said front and back sides together.

4) The device of claim 1, wherein article of clothing comprises a lower body covering having a first and second leg portion and a torso portion.

5) The device of claim 4, wherein:
said lower body leg portions further comprise separated front and back sides having disconnected edges therebetween,
said disconnected edges having a strap and strap connection means for securing said front and back sides together.

6) The device of claim 1, wherein article of clothing comprises shoe coverings having a shoe upper covering.

7) The device of claim 6, wherein:
said shoe coverings further comprise a rear slit;
said rear slit having a strap and strap connection means for securing said front and back sides together.

8) The device of claim 1, wherein article of clothing comprises an upper body covering having a front side and a back side, said front and back sides connected by a first and second shoulder portion, and a first and second arm covering portion.

9) The device of claim 8, wherein:
said upper body covering and arm covering portions further comprising separated front and back sides having disconnected edges therebetween,
said disconnected edges having a strap and strap connection means for securing said front and back sides together.

10) The device of claim 1, wherein article of clothing comprises a headband having an elongated body portion adapted to encircle a user's head.

11) The device of claim 10, wherein:
said headband elongated body further comprises a rear slit having disconnected edges therebetween,
said disconnected edges having a strap and strap connection means for securing said front and back sides together.

12) The device of claim 1, wherein article of clothing comprises a shoe sole having an elongated body portion adapted to insert into a user's footwear.

13) The device of claim 1, wherein article of clothing comprises a hand covering having an elongated body portion and finger portions adapted to cover a user's hand.

14) The device of claim 1, wherein said air-activated heating compound further comprises iron powder, a salt, water, an absorbent material, and activated carbon, said compound being reactive with oxygen to create an exothermic reaction.

15) The device of claim 1, further comprising a vacuum sealed wrapping for storing said garment prior to use, whereby said wrapping comprises an interior volume to accept said garment and a sealed opening.
